(19) United States
(12) Patent Application Publication
(10) Pub. No.: US 2024/0118213 A1
(43) Pub. Date: Apr. 11, 2024

(54) MEASURING DRILLING FLUID SALINITY WITH SMART POLYMERS

(71) Applicant: Saudi Arabian Oil Company, Dhahran (SA)

(72) Inventors: Mohammed Albassam, Alkhobar (SA); Arturo Magana Mora, Dhahran (SA); Chinthaka Pasan Gooneratne, Dhahran (SA); Mohammad Aljubran, Sayhat (SA); Peter Boul, Houston, TX (US)

(21) Appl. No.: 17/960,355

(22) Filed: Oct. 5, 2022

(51) Int. Cl. G01N 21/78; E21B 21/06; G01N 33/18; G01N 33/28; G06T 7/00

(52) U.S. Cl. CPC G01N 21/78; E21B 21/062; E21B 21/065; G01N 33/182; G01N 33/2823; G06T 7/0004; G06T 2207/20081

(57) ABSTRACT

Systems and methods include techniques for using smart polymers. Units of smart polymers with chloride ion sensitivity are inserted into drilling fluid pumped into a well. The smart polymers are configured to be triggered by chlorine ion concentrations. An insertion timestamp associated with each unit is stored. Each insertion timestamp indicates a time that each unit was inserted. Continuous images and observed characteristics of returning mud exiting through an annulus of the well and containing the units of smart polymers are captured by a camera positioned at a sensing location and linked to the monitoring system. An estimate of salinity in the drilling fluid is determined using continuous images, observed characteristics, and insertion timestamps, and based at least in part on executing image processing algorithms, machine-learning models, and deep-learning models. Changes to drilling parameters are suggested based on the estimate of the salinity.

### TECHNICAL FIELD

The present disclosure applies to measuring and estimating conditions while drilling wells, e.g., oil wells.

### BACKGROUND

When drilling wells in the oil and gas industry, salinity is one of the properties that exist in drilling mud that returns from the drilling operation and is to be monitored during the drilling process. Salinity refers to the amount of salt dissolved in a body of water or fluid (e.g., drilling fluid). Salinity may typically be measured as a fraction of grams of salt per kilogram of sea water, such as in

$$\\ \frac{g salt}{kg sea water} .$$

However, in the oil and gas industry, salinity is typically measured in parts per million (ppm). Monitoring the salinity of drilling fluids is essential to ensure that the measurements are within an accepted salinity range ideal for maintaining safe and productive operations. In some cases, rocks containing water-reactive clays may swell when coming in contact with fresh water or low salinity drilling fluids. For instance, shale formations can tend to swell when low salinity drilling fluids are used. Such a resulting shale instability can lead to several undesirable events including: tight spots, stuck pipe incidents, hole cavings, drilling fluid loss circulation, cement failure, and total wellbore collapse.

Stuck pipe is an extremely costly incident that may occur during drilling. Stuck pipe incidents account for the majority of lost time during drilling operations. As the shale swells when in contact with drilling fluids with low chloride [Cl⁻] concentrations, the annular clearance for the pipe to move freely reduces and tight spots may occur. Sometimes the driller is capable of “working” these tight spots and successfully avoids getting stuck. However, at other times the shale can swell to a limit where a mechanical stuck is inevitable.

High chloride [Cl⁻] concentrations may result in developing scale downhole. Scale is an assemblage of deposits that can cake casing, production tubing, valves, pumps, and the wellbore. Scale can develop in formation pores near the wellbore, which may: 1) reduce formation porosity and permeability, 2) block flow by clogging perforations or forming a thick lining in production tubing, and 3) coat and damage downhole equipment.

Monitoring chloride concentration is also critical in cementing operations. High chloride concentrations can lead to immature cement settling that may jeopardize the entire well. Continuous measuring of drilling fluid salinity can inform the crew of the amount of space needed ahead of pumping cement. High volumes of spacers may be needed when the drilling fluid is highly saline in order to ensure that the chloride content does not interact with the cement. Continuous measurements of chloride [Cl−] concentrations can also aid in water kick detection. Brine-bearing formations are typically saline at depth.

### SUMMARY

The present disclosure describes techniques for using smart polymers and a camera positioned in a well (e.g., at a shale shaker) to estimate salinity of drilling fluid in a wellbore. In some implementations, a computer-implemented method includes the following. Units of smart polymers with chloride ion sensitivity are inserted by a monitoring system into drilling fluid pumped into a well during a drilling operation. The smart polymers are configured to be triggered by chlorine ion concentrations. An insertion timestamp associated with each unit is stored by the monitoring system. Each insertion timestamp indicates a time that each unit was inserted into the drilling fluid. Continuous images and observed characteristics of returning mud exiting through an annulus of the well and containing the units of smart polymers are captured by a camera positioned at a sensing location and linked to the monitoring system. An estimate of salinity in the drilling fluid at a drill bit of the drilling operation is determined by the monitoring system using the continuous images, the observed characteristics, and the insertion timestamps associated with each unit of smart polymer. Determining the estimate is based at least in part on executing image processing algorithms, machine-learning models, and deep-learning models. Changes to be made to drilling parameters for the drilling operation are suggested by the monitoring system based at least in part on the estimate of the salinity of the drilling fluid.

The previously described implementation is implementable using a computer-implemented method; a non-transitory, computer-readable medium storing computer-readable instructions to perform the computer-implemented method; and a computer-implemented system including a computer memory interoperably coupled with a hardware processor configured to perform the computer-implemented method, the instructions stored on the non-transitory, computer-readable medium.

The subject matter described in this specification can be implemented in particular implementations, so as to realize one or more of the following advantages. A camera at the shale shaker can be used to help identify unplanned reductions or increases in salinity that may lead to either fresh or saline water detection. Such identification can be useful in oil drilling operations since shale instability is one of the main reasons for tight holes and stuck pipe incidents. In addition, shale instability may also result in loss of circulation, which may then lead to well control incidents. The techniques of the present disclosure can also aid in water kick detection. Current limitations with drilling fluid salinity measurements can be addressed. Typically, full drilling fluid checks with determination of salinity are only performed twice a day. Such intermittent and unreliable measurements are not sufficient to provide the required inputs for near “real time” fluid checks and control. Moreover, the techniques of the present disclosure can take advantage of emerging technologies aligned with the fourth industrial revolution (4IR), such as automation, Internet of Things (IoT), artificial intelligence (AI) machine learning, and data analytics. Techniques of the present disclosure can utilize a camera at the shale shaker and smart polymers to evaluate the drilling fluids salinity at different timestamps to ensure the salinity of the fluid is within the acceptable range for safe operations.

The details of one or more implementations of the subject matter of this specification are set forth in the Detailed Description, the accompanying drawings, and the claims. Other features, aspects, and advantages of the subject matter will become apparent from the Detailed Description, the claims, and the accompanying drawings.

### DESCRIPTION OF DRAWINGS

FIG. 1 is a plan view of an example of a shale shaker configuration, according to some implementations of the present disclosure.

FIG. 2 is a drawing showing an example of inputs and outputs of a system for estimating drilling fluid salinity, according to some implementations of the present disclosure.

FIG. 3 is a diagram showing an example of a supervised learning method to estimate drilling fluid salinity, according to some implementations of the present disclosure.

FIG. 4 is a flowchart of an example of a method for estimating drilling fluid salinity, according to some implementations of the present disclosure.

FIG. 5 is a block diagram illustrating an example computer system used to provide computational functionalities associated with described algorithms, methods, functions, processes, flows, and procedures as described in the present disclosure, according to some implementations of the present disclosure.

Like reference numbers and designations in the various drawings indicate like elements.

### DETAILED DESCRIPTION

The following detailed description describes techniques for estimating salinity at a drill bit during a drilling operation through the use of smart polymers introduced into drilling fluid and photographed in returning drilling mud after exposure to downhole conditions. Various modifications, alterations, and permutations of the disclosed implementations can be made and will be readily apparent to those of ordinary skill in the art, and the general principles defined may be applied to other implementations and applications, without departing from the scope of the disclosure. In some instances, details unnecessary to obtain an understanding of the described subject matter may be omitted so as to not obscure one or more described implementations with unnecessary detail and inasmuch as such details are within the skill of one of ordinary skill in the art. The present disclosure is not intended to be limited to the described or illustrated implementations, but to be accorded the widest scope consistent with the described principles and features.

The present disclosure includes a method for utilizing smart fluids/polymers and a camera at the shale shaker to measure the salinity of drilling fluids in the wellbore. Smart polymers are stimuli-responsive polymers that change properties according to the environment in which they are placed. Different stimuli can include pressure, temperature, and concentration of hydrogen (pH) (or ionic strength). Changes in properties can include shape, chemical properties, or even color. The present disclosure focuses on salinity as a trigger to changes in color of smart polymers/fluids detected by the camera at the shale shaker.

The continuous measurement of the drilling fluid salinity is an essential task to ensure a safe drilling environment. Consequently, the present disclosure describes the use of smart polymers that react to chloride [Cl⁻] concentrations stimuli in order to automatically estimate the salinity of drilling fluid. The smart polymers can be used with an Internet of things (IoT) platform at a drilling rig. The platform can include: 1) smart, waterproof, high-resolution, wireless cameras, or any other image or vision sensor, including infrared, gamma ray, computerized tomography (CT) scan, and x-ray, among others, for image/video capture; 2) an edge/fog computing hardware; and 3) software for image/video processing and artificial intelligence algorithms to automatically estimate the salinity values from observed polymer characteristics at the shale shaker to transform discrete images to digital salinity measurements.

While cameras have been used in the industry to monitor the drilling mud returns at shale shakers, the use of smart polymers along with artificial intelligence models to detect and quantify certain conditions remains a novel application. Smart polymers are stimuli-responsive polymers that change properties according to the environment in which they are placed. Different stimuli include temperature, pH, or ionic strength, among others. Changes in properties include changing shape, chemical properties, emitting light, or color. The present disclosure describes the use of chloride [Cl⁻] concentration as a trigger to change the color/intensity of smart polymers/fluids that can be automatically detected by computational models to analyze the frames obtained by the camera recording the shale shaker.

Types of stimuli-responsive polymers include reversible and irreversible. Irreversible polymers do not return to their natural state once the trigger has been eliminated from the environment. The present disclosure focuses on reversible polymers where properties do return back to their initial state. The use of reversible polymers allows the system to measure salinity at different times and locations in the fluid column.

The present disclosure addresses the current limitations with drilling fluid salinity measurements. In conventional systems, full drilling fluid check with a determination of salinity is only performed twice a day at most. Such intermittent and unreliable measurements are not sufficient to provide the required inputs for near “real time” fluid checks and control. The near real-time salinity checks of the present disclosure can ensure that the drilling fluid remains in the required salinity range window for it to be effective.

In the techniques described in the present disclosure, the smart polymers (e.g., in the form of pills of various sizes and shapes) are designed to be pumped with the drilling fluid used during drilling operations. As an example, the pills can be pumped with the drilling mud at different intervals (e.g., every one, three or five minutes). In another example, the pills can be pumped every one stand (e.g., every 90 feet). In this way, the smart polymers delivered as pills can serve as “salinity polymers” that are designed to be triggered by mechanical stress. For example, the salinity-responsive polymers can change properties as a function of the chloride [Cl−] concentration in the drilling fluid. As smart polymer pills exit the well through the annulus, for example, the camera at the shale shaker can capture continuous images of the returning mud. Image processing algorithms as well as machine-learning (ML) and deep-learning (DL) models can be used to predict/estimate salinity measurements.

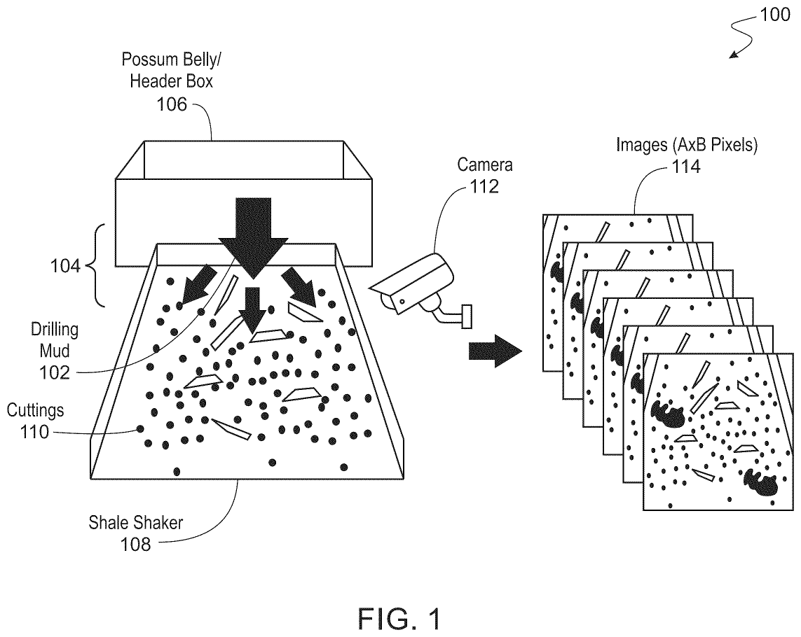

FIG. 1 is a plan view of an example of a shale shaker configuration 100, according to some implementations of the present disclosure. Drilling mud flow 102 direction is represented by arrows 104. The mud enters a solids control process from a possum belly/header box 106. In this example, gravity feeds the mud into the vibrating basket of a shale shaker 108, loaded with course and fine mesh screens designed to sort the solids (e.g., cuttings 110) from the liquid phase. The mud moves from top to bottom, as shown in FIG. 1, through a motion caused by shaker basket vibration. As the drilling mud travels, the vibrational impact with the screen causes liquid/solid separation and/or drying. Upon discharge at the bottom of the shale shaker 108, the solids are discarded (as shown) while the liquid (and fine solids, depending on screen size) pass into the sump tank for further treatment and ultimate recycling for re-pumping downhole. A camera 112 is used to capture images 114, e.g., of dimension size A×B. The images 114 are processed by image processing algorithms and ML to convert analog data (e.g., intensity, color, and light) to numerical salinity values. Vision sensing can occur at multiple locations using multiple cameras, for example, at solids discharge from one or more of the shale shaker, centrifuges, de-sanders, de-silters, and locations using other solids control technologies. However, the present disclosure focuses on the shale shaker, with surface screening of solids in a load and discharge configuration as shown in FIG. 1.

### Chemical Detection of Chloride

The detection of chloride in drilling muds can be achieved through the inclusion of a fluorescent probe sensing capability based on dual emissive carbon dots (CDs). The CDs can be synthesized by one-pot solvothermal treatment of o-phenylenediamine (OPDA), for example. The ratiometric fluorescence intensity (e.g., F410 nanometers (nm_ and F568 nm) of the synthesized CDs can be used for the quantification of Cl−.

Alternatively, Polyethyleneimine(PEI)-capped Ag nanoclusters are also a fluorescent and colorimetric platform which can be used for the sensitive and selective detection of halide ions. This type of sensing system can exhibits a remarkably high selectivity toward halide ions over most anions and cations. As an example, high sensitivity to iodide and bromide in coexistence with chloride can be achieved with high ionic strength solutions.

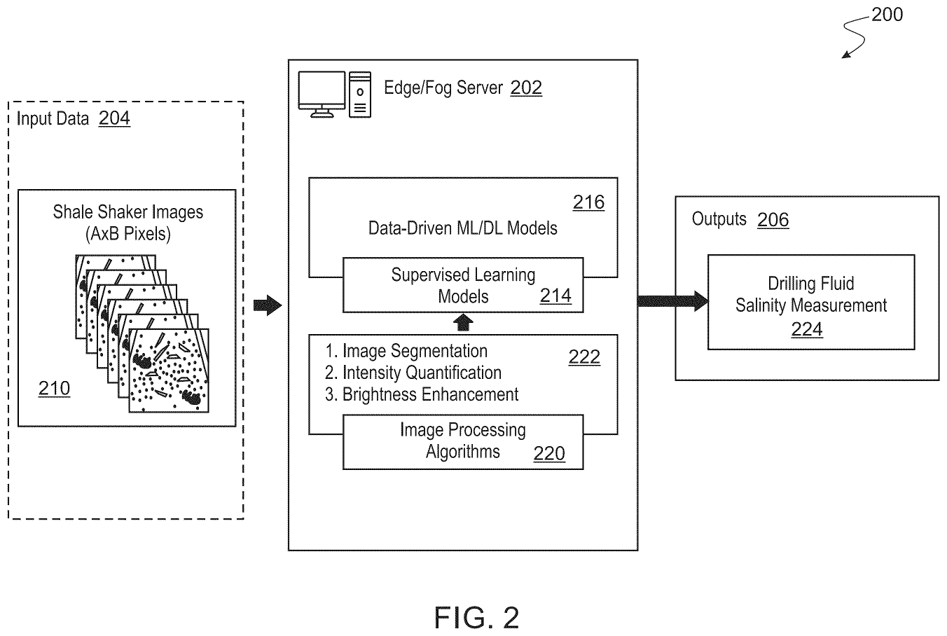

The camera at the shale shaker along with chloride ion sensitive polymers offer a new method to measure salinity. The novelty of the present disclosure is not in the polymer formulation, as these are well established in the literature and science. The novelty of the present disclosure is provided by using these chloride-responsive polymers to measure returning drilling fluids' salinity values in the oil and gas industry. In addition, techniques of the present disclosure can use ML/DL models that are capable of estimating/quantifying salinity values using image data obtained at the shale shakers. Consequently, the methods described in the present disclosure have the application of continuous measurements of drilling fluids salinity measurement (FIG. 2)

FIG. 2 is a drawing showing an example of inputs and outputs of a system 200 for estimating drilling fluid salinity, according to some implementations of the present disclosure. The system 200 can be used for the estimation of salinity, operational margins, and hole cleaning performance. The system 200 includes an edge/fog server 202 that processes input data 204 for the system 200 and generates outputs 206, including a drilling fluid salinity measurement 224.

The input data 204 can include drilling parameters and sensor data and image data (e.g., shale shaker images 210). The drilling parameters and sensor data 208 can include depth data, mud flow rates, mud rheology, stand pipe pressures, and pressures (PWD).

The edge/fog server 202 can use various models, including supervised learning models 214, which can serve as inputs to data-driven ML/DL models 216. The physics-based models 212 can use as input ECD data. The supervised learning models 214 can use as input outputs of image processing algorithms 220 that can perform functions 222 including image segmentations, intensity quantification, and brightness enhancement.

Image data from the camera/vision sensors are expected to be primarily processed in continuous recording to capture the trends of the flow over time. The frames from the camera can be processed by the image processing algorithms 220 and the ML/DL models 216 deployed in the edge/fog server 202. The methods described in the present disclosure use a set of image processing techniques to detect polymer features (e.g., the polymer intensity, color, and light) in the frame as well as to enhance the contrast and brightness of the frames. These image processing techniques can include pixilation, image segmentation, intensity quantification, or supervised learning models (e.g., including ML and DL). The algorithms can convert the images to arrays (e.g., multi-dimensional arrays) that can later be translated to numerical salinity values. The numerical representation of the intensities of the polymers mixed with the fluid observed at the shale shakers can be directly used to estimate the salinity values. For instance, a simple logistic regression model may be used as follows:

Salinity=β×max(pixel intensity), (1)

where β refers to the coefficient learned by the regression model and max(pixel intensity) to the pixel with the highest intensity values in a frame, respectively. Salinity refers to the salinity measurements as recorded in the laboratory. The linear regression, as a supervised learning model, can learn this relationship by observing multiple samples S with their respective target labels (salinity).

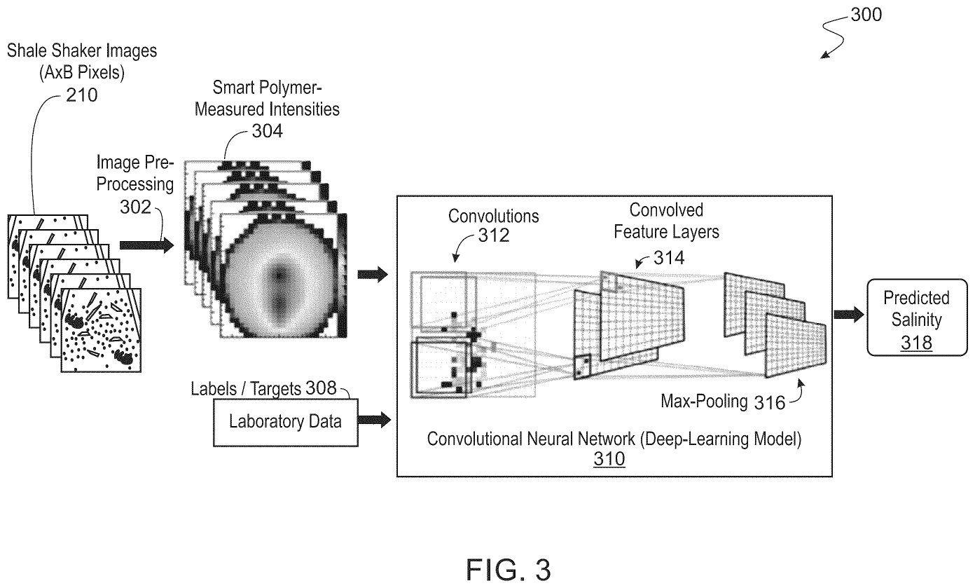

However, relying on the value of a single pixel intensity (capturing the polymer activation) may not provide enough accuracy. Consequently, supervised learning DL models, such as convolutional neural networks (CNN), and auto encoder neural networks (AE-NN), among others, can be derived from the frames to classify the observed (intensity/color/light) images. These DL models can automatically extract abstract features from the frames that can be linked to the chloride [Cl⁻] concentration as a target. In supervised learning, each frame containing the set of intensities observed from the smart polymer can be assigned a label (salinity value) to train the regression DL model, as shown in FIG. 3.

To label the data (frames with their respective chloride concentration), the smart polymers pills can be exposed to different chloride [Cl] concentrations in the lab, and the values can be recorded using a salinity measurement tool kit. Certain salinity values can be applied, and images of the resulting polymers can be acquired. These images, along with the actual applied salinity values, can then be used to train the machine model. Notably, laboratory data is to be required for the learning phase of the model, as the laboratory data represents targets/labels. After the model is derived, the objective of the model is to predict these values based on images alone.

FIG. 3 is a diagram showing an example of a supervised learning method 300 to estimate drilling fluid salinity, according to some implementations of the present disclosure. The supervised learning method 300 can be used as initial input for the shale shaker images 210. Image pre-processing 302 performed on the shale shaker images 210 can create smart polymer measured intensities 304. Labels/targets 308, along with the smart polymer measured intensities 304, can serve as inputs to a Convolutional Neural Network (CNN) (e.g., deep learning model) 310. Convolutions 312 can be used to create convolved feature layers 314 from which max-pooling 316 is performed. Output of the CNN 310 is predicted salinity 318 in drilling fluid.

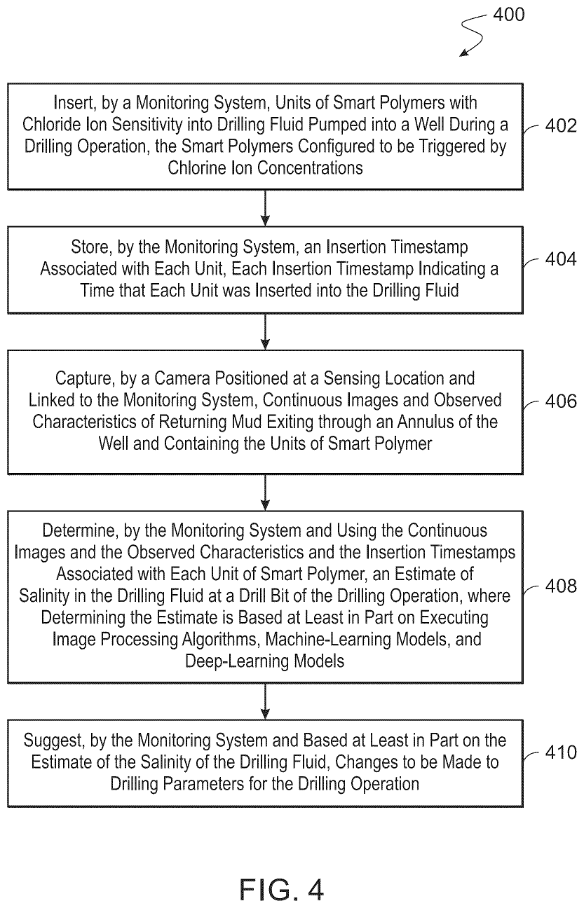

FIG. 4 is a flowchart of an example of a method 400 for estimating drilling fluid salinity, according to some implementations of the present disclosure. For clarity of presentation, the description that follows generally describes method 400 in the context of the other figures in this description. However, it will be understood that method 400 can be performed, for example, by any suitable system, environment, software, and hardware, or a combination of systems, environments, software, and hardware, as appropriate. In some implementations, various steps of method 400 can be run in parallel, in combination, in loops, or in any order.

At 402, units of smart polymers with chloride ion sensitivity are inserted by a monitoring system into drilling fluid pumped into a well during a drilling operation. The smart polymers are configured to be triggered by chlorine ion concentrations. The units of smart polymers can have a pill shape, for example. The units of smart polymers can be configured to change properties as a function of chloride ion sensitivity of the drilling fluid in downhole conditions. In some implementations, pumping the units of smart polymers into the drilling fluid can occur at different intervals (e.g., every one, three, or five minutes) or can be pumped every one stand (e.g., every 90 feet). From 402, method 400 proceeds to 404.

At 404, an insertion timestamp associated with each unit is stored by the monitoring system. Each insertion timestamp indicates a time that each unit was inserted into the drilling fluid. From 404, method 400 proceeds to 406.

At 406, continuous images and observed characteristics of returning mud exiting through an annulus of the well and containing the units of smart polymers are captured by a camera positioned at a sensing location and linked to the monitoring system. Capturing the continuous images can include capturing, in the units of smart polymers, changes in salinity identified by the smart polymers. The sensing location can be, for example, a shale shaker, a centrifuge, a de-sander, and a de-silter. From 406, method 400 proceeds to 408.

At 408, an estimate of salinity in the drilling fluid at a drill bit of the drilling operation is determined by the monitoring system using the continuous images, the observed characteristics, and the insertion timestamps associated with each unit of smart polymer. Determining the estimate is based at least in part on executing image processing algorithms, machine-learning models, and deep-learning models. In some implementations, estimating the salinity can include correlating an arrival timestamp identifying a time of arrival of each unit of smart polymer at the sensing location with a respective hole depth by utilizing a rig sensor for mud flow rate and based on an annular area of the well. From 408, method 400 proceeds to 410.

At 410, changes to be made to drilling parameters for the drilling operation are suggested by the monitoring system based at least in part on the estimate of the salinity of the drilling fluid. For example, changes can be made in drilling parameters that are associated with changes in mud rheology, mud weight, and mud flow rate. After 410, method 400 can stop.

In some implementations, in addition to (or in combination with) any previously-described features, techniques of the present disclosure can include the following. Outputs of the techniques of the present disclosure can be performed before, during, or in combination with wellbore operations, such as to provide inputs to change the settings or parameters of equipment used for drilling. Examples of wellbore operations include forming/drilling a wellbore, hydraulic fracturing, and producing through the wellbore, to name a few. The wellbore operations can be triggered or controlled, for example, by outputs of the methods of the present disclosure. In some implementations, customized user interfaces can present intermediate or final results of the above described processes to a user. Information can be presented in one or more textual, tabular, or graphical formats, such as through a dashboard. The information can be presented at one or more on-site locations (such as at an oil well or other facility), on the Internet (such as on a webpage), on a mobile application (or “app”), or at a central processing facility. The presented information can include suggestions, such as suggested changes in parameters or processing inputs, that the user can select to implement improvements in a production environment, such as in the exploration, production, and/or testing of petrochemical processes or facilities. For example, the suggestions can include parameters that, when selected by the user, can cause a change to, or an improvement in, drilling parameters (including drill bit speed and direction) or overall production of a gas or oil well. The suggestions, when implemented by the user, can improve the speed and accuracy of calculations, streamline processes, improve models, and solve problems related to efficiency, performance, safety, reliability, costs, downtime, and the need for human interaction. In some implementations, the suggestions can be implemented in real-time, such as to provide an immediate or near-immediate change in operations or in a model. The term real-time can correspond, for example, to events that occur within a specified period of time, such as within one minute or within one second. Events can include readings or measurements captured by downhole equipment such as sensors. The readings or measurements can be analyzed at the surface, such as by using applications that can include modeling applications and machine learning. The analysis can be used to generate changes to settings of downhole equipment, such as drilling equipment. In some implementations, values of parameters or other variables that are determined can be used automatically (such as through using rules) to implement changes in oil or gas well exploration, production/drilling, or testing. For example, outputs of the present disclosure can be used as inputs to other equipment and/or systems at a facility. This can be especially useful for systems or various pieces of equipment that are located several meters or several miles apart, or are located in different countries or other jurisdictions.

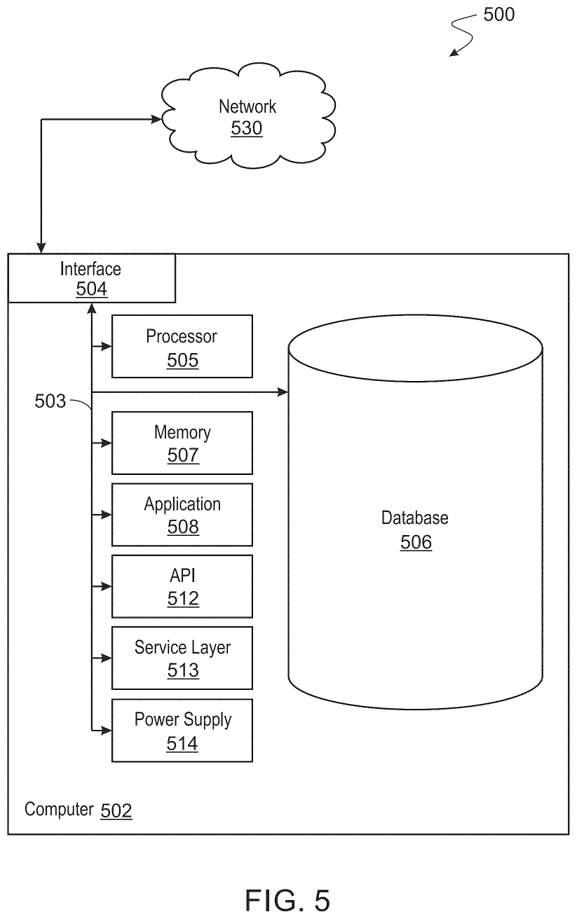

FIG. 5 is a block diagram of an example computer system 500 used to provide computational functionalities associated with described algorithms, methods, functions, processes, flows, and procedures described in the present disclosure, according to some implementations of the present disclosure. The illustrated computer 502 is intended to encompass any computing device such as a server, a desktop computer, a laptop/notebook computer, a wireless data port, a smart phone, a personal data assistant (PDA), a tablet computing device, or one or more processors within these devices, including physical instances, virtual instances, or both. The computer 502 can include input devices such as keypads, keyboards, and touch screens that can accept user information. Also, the computer 502 can include output devices that can convey information associated with the operation of the computer 502. The information can include digital data, visual data, audio information, or a combination of information. The information can be presented in a graphical user interface (UI) (or GUI).

The computer 502 can serve in a role as a client, a network component, a server, a database, a persistency, or components of a computer system for performing the subject matter described in the present disclosure. The illustrated computer 502 is communicably coupled with a network 530. In some implementations, one or more components of the computer 502 can be configured to operate within different environments, including cloud-computing-based environments, local environments, global environments, and combinations of environments.

At a top level, the computer 502 is an electronic computing device operable to receive, transmit, process, store, and manage data and information associated with the described subject matter. According to some implementations, the computer 502 can also include, or be communicably coupled with, an application server, an email server, a web server, a caching server, a streaming data server, or a combination of servers.

The computer 502 can receive requests over network 530 from a client application (for example, executing on another computer 502). The computer 502 can respond to the received requests by processing the received requests using software applications. Requests can also be sent to the computer 502 from internal users (for example, from a command console), external (or third) parties, automated applications, entities, individuals, systems, and computers.

Each of the components of the computer 502 can communicate using a system bus 503. In some implementations, any or all of the components of the computer 502, including hardware or software components, can interface with each other or the interface 504 (or a combination of both) over the system bus 503. Interfaces can use an application programming interface (API) 512, a service layer 513, or a combination of the API 512 and service layer 513. The API 512 can include specifications for routines, data structures, and object classes. The API 512 can be either computer-language independent or dependent. The API 512 can refer to a complete interface, a single function, or a set of APIs.

The service layer 513 can provide software services to the computer 502 and other components (whether illustrated or not) that are communicably coupled to the computer 502. The functionality of the computer 502 can be accessible to all service consumers using this service layer. Software services, such as those provided by the service layer 513, can provide reusable, defined functionalities through a defined interface. For example, the interface can be software written in JAVA, C++, or a language providing data in extensible markup language (XML) format. While illustrated as an integrated component of the computer 502, in alternative implementations, the API 512 or the service layer 513 can be stand-alone components in relation to other components of the computer 502 and other components communicably coupled to the computer 502. Moreover, any or all parts of the API 512 or the service layer 513 can be implemented as child or sub-modules of another software module, enterprise application, or hardware module without departing from the scope of the present disclosure.

The computer 502 includes an interface 504. Although illustrated as a single interface 504 in FIG. 5, two or more interfaces 504 can be used according to particular needs, desires, or particular implementations of the computer 502 and the described functionality. The interface 504 can be used by the computer 502 for communicating with other systems that are connected to the network 530 (whether illustrated or not) in a distributed environment. Generally, the interface 504 can include, or be implemented using, logic encoded in software or hardware (or a combination of software and hardware) operable to communicate with the network 530. More specifically, the interface 504 can include software supporting one or more communication protocols associated with communications. As such, the network 530 or the interface's hardware can be operable to communicate physical signals within and outside of the illustrated computer 502.

The computer 502 includes a processor 505. Although illustrated as a single processor 505 in FIG. 5, two or more processors 505 can be used according to particular needs, desires, or particular implementations of the computer 502 and the described functionality. Generally, the processor 505 can execute instructions and can manipulate data to perform the operations of the computer 502, including operations using algorithms, methods, functions, processes, flows, and procedures as described in the present disclosure.

The computer 502 also includes a database 506 that can hold data for the computer 502 and other components connected to the network 530 (whether illustrated or not). For example, database 506 can be an in-memory, conventional, or a database storing data consistent with the present disclosure. In some implementations, database 506 can be a combination of two or more different database types (for example, hybrid in-memory and conventional databases) according to particular needs, desires, or particular implementations of the computer 502 and the described functionality. Although illustrated as a single database 506 in FIG. 5, two or more databases (of the same, different, or combination of types) can be used according to particular needs, desires, or particular implementations of the computer 502 and the described functionality. While database 506 is illustrated as an internal component of the computer 502, in alternative implementations, database 506 can be external to the computer 502.

The computer 502 also includes a memory 507 that can hold data for the computer 502 or a combination of components connected to the network 530 (whether illustrated or not). Memory 507 can store any data consistent with the present disclosure. In some implementations, memory 507 can be a combination of two or more different types of memory (for example, a combination of semiconductor and magnetic storage) according to particular needs, desires, or particular implementations of the computer 502 and the described functionality. Although illustrated as a single memory 507 in FIG. 5, two or more memories 507 (of the same, different, or combination of types) can be used according to particular needs, desires, or particular implementations of the computer 502 and the described functionality. While memory 507 is illustrated as an internal component of the computer 502, in alternative implementations, memory 507 can be external to the computer 502.

The application 508 can be an algorithmic software engine providing functionality according to particular needs, desires, or particular implementations of the computer 502 and the described functionality. For example, application 508 can serve as one or more components, modules, or applications. Further, although illustrated as a single application 508, the application 508 can be implemented as multiple applications 508 on the computer 502. In addition, although illustrated as internal to the computer 502, in alternative implementations, the application 508 can be external to the computer 502.

The computer 502 can also include a power supply 514. The power supply 514 can include a rechargeable or non-rechargeable battery that can be configured to be either user- or non-user-replaceable. In some implementations, the power supply 514 can include power-conversion and management circuits, including recharging, standby, and power management functionalities. In some implementations, the power supply 514 can include a power plug to allow the computer 502 to be plugged into a wall socket or a power source to, for example, power the computer 502 or recharge a rechargeable battery.

There can be any number of computers 502 associated with, or external to, a computer system containing computer 502, with each computer 502 communicating over network 530. Further, the terms “client,” “user,” and other appropriate terminology can be used interchangeably, as appropriate, without departing from the scope of the present disclosure. Moreover, the present disclosure contemplates that many users can use one computer 502 and one user can use multiple computers 502.

Described implementations of the subject matter can include one or more features, alone or in combination.

For example, in a first implementation, a computer-implemented method includes the following. Units of smart polymers with chloride ion sensitivity are inserted by a monitoring system into drilling fluid pumped into a well during a drilling operation. The smart polymers are configured to be triggered by chlorine ion concentrations. An insertion timestamp associated with each unit is stored by the monitoring system. Each insertion timestamp indicates a time that each unit was inserted into the drilling fluid. Continuous images and observed characteristics of returning mud exiting through an annulus of the well and containing the units of smart polymers are captured by a camera positioned at a sensing location and linked to the monitoring system. An estimate of salinity in the drilling fluid at a drill bit of the drilling operation is determined by the monitoring system using the continuous images, the observed characteristics, and the insertion timestamps associated with each unit of smart polymer. Determining the estimate is based at least in part on executing image processing algorithms, machine-learning models, and deep-learning models. Changes to be made to drilling parameters for the drilling operation are suggested by the monitoring system based at least in part on the estimate of the salinity of the drilling fluid.

The foregoing and other described implementations can each, optionally, include one or more of the following features:
A first feature, combinable with any of the following features, where the units of smart polymers have a pill shape.
A second feature, combinable with any of the previous or following features, where the method further includes pumping the units of smart polymers into the drilling fluid at different intervals or pumped every one stand.
A third feature, combinable with any of the previous or following features, where the units of smart polymers are configured to change properties as a function of chloride ion sensitivity of the drilling fluid in downhole conditions.
A fourth feature, combinable with any of the previous or following features, where capturing the continuous images includes capturing, in the units of smart polymers, changes in salinity identified by the smart polymers.
A fifth feature, combinable with any of the previous or following features, where estimating the salinity includes correlating an arrival timestamp identifying a time of arrival of each unit of smart polymer at the sensing location with a respective hole depth by utilizing a rig sensor for mud flow rate and based on an annular area of the well.
A sixth feature, combinable with any of the previous or following features, where the sensing location is selected from the group consisting of a shale shaker, a centrifuge, a de-sander, and a de-silter.

In a second implementation, a non-transitory, computer-readable medium stores one or more instructions executable by a computer system to perform operations including the following. Units of smart polymers with chloride ion sensitivity are inserted by a monitoring system into drilling fluid pumped into a well during a drilling operation. The smart polymers are configured to be triggered by chlorine ion concentrations. An insertion timestamp associated with each unit is stored by the monitoring system. Each insertion timestamp indicates a time that each unit was inserted into the drilling fluid. Continuous images and observed characteristics of returning mud exiting through an annulus of the well and containing the units of smart polymers are captured by a camera positioned at a sensing location and linked to the monitoring system. An estimate of salinity in the drilling fluid at a drill bit of the drilling operation is determined by the monitoring system using the continuous images, the observed characteristics, and the insertion timestamps associated with each unit of smart polymer. Determining the estimate is based at least in part on executing image processing algorithms, machine-learning models, and deep-learning models. Changes to be made to drilling parameters for the drilling operation are suggested by the monitoring system based at least in part on the estimate of the salinity of the drilling fluid.

The foregoing and other described implementations can each, optionally, include one or more of the following features:
A first feature, combinable with any of the following features, where the units of smart polymers have a pill shape.
A second feature, combinable with any of the previous or following features, where the operations further include pumping the units of smart polymers into the drilling fluid at different intervals or pumped every one stand.
A third feature, combinable with any of the previous or following features, where the units of smart polymers are configured to change properties as a function of chloride ion sensitivity of the drilling fluid in downhole conditions.
A fourth feature, combinable with any of the previous or following features, where capturing the continuous images includes capturing, in the units of smart polymers, changes in salinity identified by the smart polymers.
A fifth feature, combinable with any of the previous or following features, where estimating the salinity includes correlating an arrival timestamp identifying a time of arrival of each unit of smart polymer at the sensing location with a respective hole depth by utilizing a rig sensor for mud flow rate and based on an annular area of the well.
A sixth feature, combinable with any of the previous or following features, where the sensing location is selected from the group consisting of a shale shaker, a centrifuge, a de-sander, and a de-silter.

In a third implementation, a computer-implemented system includes one or more processors and a non-transitory computer-readable storage medium coupled to the one or more processors and storing programming instructions for execution by the one or more processors. The programming instructions instruct the one or more processors to perform operations including the following. Units of smart polymers with chloride ion sensitivity are inserted by a monitoring system into drilling fluid pumped into a well during a drilling operation. The smart polymers are configured to be triggered by chlorine ion concentrations. An insertion timestamp associated with each unit is stored by the monitoring system. Each insertion timestamp indicates a time that each unit was inserted into the drilling fluid. Continuous images and observed characteristics of returning mud exiting through an annulus of the well and containing the units of smart polymers are captured by a camera positioned at a sensing location and linked to the monitoring system. An estimate of salinity in the drilling fluid at a drill bit of the drilling operation is determined by the monitoring system using the continuous images, the observed characteristics, and the insertion timestamps associated with each unit of smart polymer. Determining the estimate is based at least in part on executing image processing algorithms, machine-learning models, and deep-learning models. Changes to be made to drilling parameters for the drilling operation are suggested by the monitoring system based at least in part on the estimate of the salinity of the drilling fluid.

The foregoing and other described implementations can each, optionally, include one or more of the following features:
A first feature, combinable with any of the following features, where the units of smart polymers have a pill shape.
A second feature, combinable with any of the previous or following features, where the operations further include pumping the units of smart polymers into the drilling fluid at different intervals or pumped every one stand.
A third feature, combinable with any of the previous or following features, where the units of smart polymers are configured to change properties as a function of chloride ion sensitivity of the drilling fluid in downhole conditions.
A fourth feature, combinable with any of the previous or following features, where capturing the continuous images includes capturing, in the units of smart polymers, changes in salinity identified by the smart polymers.
A fifth feature, combinable with any of the previous or following features, where estimating the salinity includes correlating an arrival timestamp identifying a time of arrival of each unit of smart polymer at the sensing location with a respective hole depth by utilizing a rig sensor for mud flow rate and based on an annular area of the well.

Implementations of the subject matter and the functional operations described in this specification can be implemented in digital electronic circuitry, in tangibly embodied computer software or firmware, in computer hardware, including the structures disclosed in this specification and their structural equivalents, or in combinations of one or more of them. Software implementations of the described subject matter can be implemented as one or more computer programs. Each computer program can include one or more modules of computer program instructions encoded on a tangible, non-transitory, computer-readable computer-storage medium for execution by, or to control the operation of, data processing apparatus. Alternatively, or additionally, the program instructions can be encoded in/on an artificially generated propagated signal. For example, the signal can be a machine-generated electrical, optical, or electromagnetic signal that is generated to encode information for transmission to a suitable receiver apparatus for execution by a data processing apparatus. The computer-storage medium can be a machine-readable storage device, a machine-readable storage substrate, a random or serial access memory device, or a combination of computer-storage mediums.

The terms “data processing apparatus,” “computer,” and “electronic computer device” (or equivalent as understood by one of ordinary skill in the art) refer to data processing hardware. For example, a data processing apparatus can encompass all kinds of apparatuses, devices, and machines for processing data, including by way of example, a programmable processor, a computer, or multiple processors or computers. The apparatus can also include special purpose logic circuitry including, for example, a central processing unit (CPU), a field-programmable gate array (FPGA), or an application-specific integrated circuit (ASIC). In some implementations, the data processing apparatus or special purpose logic circuitry (or a combination of the data processing apparatus or special purpose logic circuitry) can be hardware- or software-based (or a combination of both hardware- and software-based). The apparatus can optionally include code that creates an execution environment for computer programs, for example, code that constitutes processor firmware, a protocol stack, a database management system, an operating system, or a combination of execution environments. The present disclosure contemplates the use of data processing apparatuses with or without conventional operating systems, such as LINUX, UNIX, WINDOWS, MAC OS, ANDROID, or IOS.

A computer program, which can also be referred to or described as a program, software, a software application, a module, a software module, a script, or code, can be written in any form of programming language. Programming languages can include, for example, compiled languages, interpreted languages, declarative languages, or procedural languages. Programs can be deployed in any form, including as stand-alone programs, modules, components, subroutines, or units for use in a computing environment. A computer program can, but need not, correspond to a file in a file system. A program can be stored in a portion of a file that holds other programs or data, for example, one or more scripts stored in a markup language document, in a single file dedicated to the program in question, or in multiple coordinated files storing one or more modules, sub-programs, or portions of code. A computer program can be deployed for execution on one computer or on multiple computers that are located, for example, at one site or distributed across multiple sites that are interconnected by a communication network. While portions of the programs illustrated in the various figures may be shown as individual modules that implement the various features and functionality through various objects, methods, or processes, the programs can instead include a number of sub-modules, third-party services, components, and libraries. Conversely, the features and functionality of various components can be combined into single components as appropriate. Thresholds used to make computational determinations can be statically, dynamically, or both statically and dynamically determined.

The methods, processes, or logic flows described in this specification can be performed by one or more programmable computers executing one or more computer programs to perform functions by operating on input data and generating output. The methods, processes, or logic flows can also be performed by, and apparatus can also be implemented as, special purpose logic circuitry, for example, a CPU, an FPGA, or an ASIC.

Computers suitable for the execution of a computer program can be based on one or more of general and special purpose microprocessors and other kinds of CPUs. The elements of a computer are a CPU for performing or executing instructions and one or more memory devices for storing instructions and data. Generally, a CPU can receive instructions and data from (and write data to) a memory.

Graphics processing units (GPUs) can also be used in combination with CPUs. The GPUs can provide specialized processing that occurs in parallel to processing performed by CPUs. The specialized processing can include artificial intelligence (AI) applications and processing, for example. GPUs can be used in GPU clusters or in multi-GPU computing.

A computer can include, or be operatively coupled to, one or more mass storage devices for storing data. In some implementations, a computer can receive data from, and transfer data to, the mass storage devices including, for example, magnetic, magneto-optical disks, or optical disks. Moreover, a computer can be embedded in another device, for example, a mobile telephone, a personal digital assistant (PDA), a mobile audio or video player, a game console, a global positioning system (GPS) receiver, or a portable storage device such as a universal serial bus (USB) flash drive.

Computer-readable media (transitory or non-transitory, as appropriate) suitable for storing computer program instructions and data can include all forms of permanent/non-permanent and volatile/non-volatile memory, media, and memory devices. Computer-readable media can include, for example, semiconductor memory devices such as random access memory (RAM), read-only memory (ROM), phase change memory (PRAM), static random access memory (SRAM), dynamic random access memory (DRAM), erasable programmable read-only memory (EPROM), electrically erasable programmable read-only memory (EEPROM), and flash memory devices. Computer-readable media can also include, for example, magnetic devices such as tape, cartridges, cassettes, and internal/removable disks. Computer-readable media can also include magneto-optical disks and optical memory devices and technologies including, for example, digital video disc (DVD), CD-ROM, DVD+/−R, DVD-RAM, DVD-ROM, HD-DVD, and BLU-RAY.

The memory can store various objects or data, including caches, classes, frameworks, applications, modules, backup data, jobs, web pages, web page templates, data structures, database tables, repositories, and dynamic information. Types of objects and data stored in memory can include parameters, variables, algorithms, instructions, rules, constraints, and references. Additionally, the memory can include logs, policies, security or access data, and reporting files. The processor and the memory can be supplemented by, or incorporated into, special purpose logic circuitry.

Implementations of the subject matter described in the present disclosure can be implemented on a computer having a display device for providing interaction with a user, including displaying information to (and receiving input from) the user. Types of display devices can include, for example, a cathode ray tube (CRT), a liquid crystal display (LCD), a light-emitting diode (LED), and a plasma monitor. Display devices can include a keyboard and pointing devices including, for example, a mouse, a trackball, or a trackpad. User input can also be provided to the computer through the use of a touchscreen, such as a tablet computer surface with pressure sensitivity or a multi-touch screen using capacitive or electric sensing. Other kinds of devices can be used to provide for interaction with a user, including to receive user feedback including, for example, sensory feedback including visual feedback, auditory feedback, or tactile feedback. Input from the user can be received in the form of acoustic, speech, or tactile input. In addition, a computer can interact with a user by sending documents to, and receiving documents from, a device that the user uses. For example, the computer can send web pages to a web browser on a user's client device in response to requests received from the web browser.

The term “graphical user interface,” or “GUI,” can be used in the singular or the plural to describe one or more graphical user interfaces and each of the displays of a particular graphical user interface. Therefore, a GUI can represent any graphical user interface, including, but not limited to, a web browser, a touch-screen, or a command line interface (CLI) that processes information and efficiently presents the information results to the user. In general, a GUI can include a plurality of user interface (UI) elements, some or all associated with a web browser, such as interactive fields, pull-down lists, and buttons. These and other UI elements can be related to or represent the functions of the web browser.

Implementations of the subject matter described in this specification can be implemented in a computing system that includes a back-end component, for example, as a data server, or that includes a middleware component, for example, an application server. Moreover, the computing system can include a front-end component, for example, a client computer having one or both of a graphical user interface or a Web browser through which a user can interact with the computer. The components of the system can be interconnected by any form or medium of wireline or wireless digital data communication (or a combination of data communication) in a communication network. Examples of communication networks include a local area network (LAN), a radio access network (RAN), a metropolitan area network (MAN), a wide area network (WAN), Worldwide Interoperability for Microwave Access (WIMAX), a wireless local area network (WLAN) (for example, using 802.11 a/b/g/n or 802.20 or a combination of protocols), all or a portion of the Internet, or any other communication system or systems at one or more locations (or a combination of communication networks). The network can communicate with, for example, Internet Protocol (IP) packets, frame relay frames, asynchronous transfer mode (ATM) cells, voice, video, data, or a combination of communication types between network addresses.

The computing system can include clients and servers. A client and server can generally be remote from each other and can typically interact through a communication network. The relationship of client and server can arise by virtue of computer programs running on the respective computers and having a client-server relationship.

Cluster file systems can be any file system type accessible from multiple servers for read and update. Locking or consistency tracking may not be necessary since the locking of exchange file system can be done at the application layer. Furthermore, Unicode data files can be different from non-Unicode data files.

While this specification contains many specific implementation details, these should not be construed as limitations on the scope of what may be claimed, but rather as descriptions of features that may be specific to particular implementations. Certain features that are described in this specification in the context of separate implementations can also be implemented, in combination, in a single implementation. Conversely, various features that are described in the context of a single implementation can also be implemented in multiple implementations, separately, or in any suitable sub-combination. Moreover, although previously described features may be described as acting in certain combinations and even initially claimed as such, one or more features from a claimed combination can, in some cases, be excised from the combination, and the claimed combination may be directed to a sub-combination or variation of a sub-combination.

Particular implementations of the subject matter have been described. Other implementations, alterations, and permutations of the described implementations are within the scope of the following claims as will be apparent to those skilled in the art. While operations are depicted in the drawings or claims in a particular order, this should not be understood as requiring that such operations be performed in the particular order shown or in sequential order, or that all illustrated operations be performed (some operations may be considered optional), to achieve desirable results. In certain circumstances, multitasking or parallel processing (or a combination of multitasking and parallel processing) may be advantageous and performed as deemed appropriate.

Moreover, the separation or integration of various system modules and components in the previously described implementations should not be understood as requiring such separation or integration in all implementations. It should be understood that the described program components and systems can generally be integrated together in a single software product or packaged into multiple software products.

Accordingly, the previously described example implementations do not define or constrain the present disclosure. Other changes, substitutions, and alterations are also possible without departing from the spirit and scope of the present disclosure.

Furthermore, any claimed implementation is considered to be applicable to at least a computer-implemented method; a non-transitory, computer-readable medium storing computer-readable instructions to perform the computer-implemented method; and a computer system including a computer memory interoperably coupled with a hardware processor configured to perform the computer-implemented method or the instructions stored on the non-transitory, computer-readable medium.

What is claimed is:

1. A computer-implemented method, comprising:
inserting, by a monitoring system, units of smart polymers with chloride ion sensitivity into drilling fluid pumped into a well during a drilling operation, the smart polymers configured to be triggered by chlorine ion concentrations;
storing, by the monitoring system, an insertion timestamp associated with each unit, each insertion timestamp indicating a time that each unit was inserted into the drilling fluid;
capturing, by a camera positioned at a sensing location and linked to the monitoring system, continuous images and observed characteristics of returning mud exiting through an annulus of the well and containing the units of smart polymer;
determining, by the monitoring system and using the continuous images and the observed characteristics and the insertion timestamps associated with each unit of smart polymer, an estimate of salinity in the drilling fluid at a drill bit of the drilling operation, wherein determining the estimate is based at least in part on executing image processing algorithms, machine-learning models, and deep-learning models; and
suggesting, by the monitoring system and based at least in part on the estimate of the salinity of the drilling fluid, changes to be made to drilling parameters for the drilling operation.

2. The computer-implemented method of claim 1, wherein the units of smart polymers have a pill shape.

3. The computer-implemented method of claim 1, further comprising:
pumping the units of smart polymers into the drilling fluid at different intervals or pumped every one stand.

4. The computer-implemented method of claim 1, wherein the units of smart polymers are configured to change properties as a function of chloride ion sensitivity of the drilling fluid in downhole conditions.

5. The computer-implemented method of claim 1, wherein capturing the continuous images includes capturing, in the units of smart polymers, changes in salinity identified by the smart polymers.

6. The computer-implemented method of claim 1, wherein estimating the salinity includes correlating an arrival timestamp identifying a time of arrival of each unit of smart polymer at the sensing location with a respective hole depth by utilizing a rig sensor for mud flow rate and based on an annular area of the well.

7. The computer-implemented method of claim 1, wherein the sensing location is selected from the group consisting of a shale shaker, a centrifuge, a de-sander, and a de-silter.

8. A non-transitory, computer-readable medium storing one or more instructions executable by a computer system to perform operations comprising:
inserting, by a monitoring system, units of smart polymers with chloride ion sensitivity into drilling fluid pumped into a well during a drilling operation, the smart polymers configured to be triggered by chlorine ion concentrations;
storing, by the monitoring system, an insertion timestamp associated with each unit, each insertion timestamp indicating a time that each unit was inserted into the drilling fluid;
capturing, by a camera positioned at a sensing location and linked to the monitoring system, continuous images and observed characteristics of returning mud exiting through an annulus of the well and containing the units of smart polymer;
determining, by the monitoring system and using the continuous images and the observed characteristics and the insertion timestamps associated with each unit of smart polymer, an estimate of salinity in the drilling fluid at a drill bit of the drilling operation, wherein determining the estimate is based at least in part on executing image processing algorithms, machine-learning models, and deep-learning models; and
suggesting, by the monitoring system and based at least in part on the estimate of the salinity of the drilling fluid, changes to be made to drilling parameters for the drilling operation.

9. The non-transitory, computer-readable medium of claim 8, wherein the units of smart polymers have a pill shape.

10. The non-transitory, computer-readable medium of claim 8, the operations further comprising:
pumping the units of smart polymers into the drilling fluid at different intervals or pumped every one stand.

11. The non-transitory, computer-readable medium of claim 8, wherein the units of smart polymers are configured to change properties as a function of chloride ion sensitivity of the drilling fluid in downhole conditions.

12. The non-transitory, computer-readable medium of claim 8, wherein capturing the continuous images includes capturing, in the units of smart polymers, changes in salinity identified by the smart polymers.

13. The non-transitory, computer-readable medium of claim 8, wherein estimating the salinity includes correlating an arrival timestamp identifying a time of arrival of each unit of smart polymer at the sensing location with a respective hole depth by utilizing a rig sensor for mud flow rate and based on an annular area of the well.

14. The non-transitory, computer-readable medium of claim 8, wherein the sensing location is selected from the group consisting of a shale shaker, a centrifuge, a de-sander, and a de-silter.

15. A computer-implemented system, comprising:
one or more processors; and
a non-transitory computer-readable storage medium coupled to the one or more processors and storing programming instructions for execution by the one or more processors, the programming instructions instructing the one or more processors to perform operations comprising:
inserting, by a monitoring system, units of smart polymers with chloride ion sensitivity into drilling fluid pumped into a well during a drilling operation, the smart polymers configured to be triggered by chlorine ion concentrations;
storing, by the monitoring system, an insertion timestamp associated with each unit, each insertion timestamp indicating a time that each unit was inserted into the drilling fluid;
capturing, by a camera positioned at a sensing location and linked to the monitoring system, continuous images and observed characteristics of returning mud exiting through an annulus of the well and containing the units of smart polymer;
determining, by the monitoring system and using the continuous images and the observed characteristics and the insertion timestamps associated with each unit of smart polymer, an estimate of salinity in the drilling fluid at a drill bit of the drilling operation, wherein determining the estimate is based at least in part on executing image processing algorithms, machine-learning models, and deep-learning models; and
suggesting, by the monitoring system and based at least in part on the estimate of the salinity of the drilling fluid, changes to be made to drilling parameters for the drilling operation.

16. The computer-implemented system of claim 15, wherein the units of smart polymers have a pill shape.

17. The computer-implemented system of claim 15, the operations further comprising:
pumping the units of smart polymers into the drilling fluid at different intervals or pumped every one stand.

18. The computer-implemented system of claim 15, wherein the units of smart polymers are configured to change properties as a function of chloride ion sensitivity of the drilling fluid in downhole conditions.

19. The computer-implemented system of claim 15, wherein capturing the continuous images includes capturing, in the units of smart polymers, changes in salinity identified by the smart polymers.

20. The computer-implemented system of claim 15, wherein estimating the salinity includes correlating an arrival timestamp identifying a time of arrival of each unit of smart polymer at the sensing location with a respective hole depth by utilizing a rig sensor for mud flow rate and based on an annular area of the well.
